(19) United States
(12) Patent Application Publication
(10) Pub. No.: US 2024/0083848 A1
(43) Pub. Date: Mar. 14, 2024

(54) PROTECTED ALKYL TRYPTAMINES AND THEIR THERAPEUTIC USES

(71) Applicant: CAAMTECH, INC., Issaquah, WA (US)

(72) Inventor: Andrew R. CHADEAYNE, Issaquah, WA (US)

(21) Appl. No.: 18/260,711

(22) PCT Filed: Jan. 7, 2022
(86) PCT No.: PCT/US2022/011521

§ 371 Date: Jul. 7, 2023

Related U.S. Application Data
(60) Provisional application No. 63/135,140, filed on Jan. 8, 2021.
(60) Provisional application No. 63/223,747, filed on Jul. 20, 2021.
(60) Provisional application No. 63/226,816, filed on Jul. 29, 2021.

(51) Int. Cl. C07D 209/16; A61K 45/06

(52) U.S. Cl. CPC C07D 209/16; A61K 45/06

(57) ABSTRACT

The disclosure relates to protected alkyl tryptamine compounds of formula (I). The disclosure relates to compositions comprising, consisting essentially of, or consisting of a compound of formula (I) and an excipient. The disclosure also relates to pharmaceutical compositions comprising a therapeutically effective amount of a compound of formula (I) where the excipient is a pharmaceutically acceptable carrier. The disclosure further relates to therapeutic uses of compounds of formula (I).

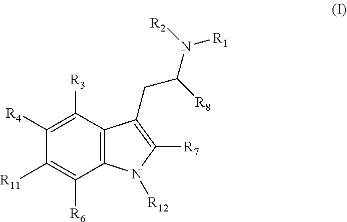

### CROSS REFERENCE TO RELATED APPLICATIONS

This application claims priority to U.S. Provisional Application No. 63/135,140, filed on Jan. 8, 2021, to U.S. Provisional Application No. 63/223,747, filed on Jul. 20, 2021, and to U.S. Provisional Application No. 63/226,816, filed on Jul. 29, 2021, the disclosures of which are incorporated by reference.

### TECHNICAL FIELD

This disclosure relates to protected alkyl tryptamines, compositions, and pharmaceutical compositions containing them as well as their use in treating various diseases.

### BACKGROUND

Psilocybin is a breakthrough drug that has received FDA approval for therapeutic applications. Psilocybin is one of several naturally occurring psychoactive tryptamines found in “magic” mushrooms. When consumed by humans, psilocybin serves as a prodrug of psilocin. Psilocin is a potent serotonin 2a-agonist, which is responsible for its psychoactive properties (Dinis-Oliveira, 2017; Nichols, 2012). Upon digestion, psilocybin hydrolyses to generate psilocin. Psychoactive tryptamines like psilocin have garnered significant interest recently because of their potential for treating mood disorders, including depression, anxiety, addiction, and post-traumatic stress disorder (PTSD) (Johnson & Griffiths, 2017; Carhart-Harris & Goodwin, 2017). Altering the chemical structure within this class of compounds can dramatically influence the potency and action of the drugs. The hydroxylation reaction used in converting psilocybin to psilocin is slow, resulting in a low bioavailability. Additionally, it is difficult and expensive to make and results in a low yield. Therefore, a better prodrug is needed to increase bioavailability.

New psychoactive tryptamines have been identified in “magic mushrooms” as recently as 2017. (Lentz, et al., 2017). Until this year, there was no general synthetic method for producing useful amounts of the minor psychoactive tryptamines. (Sherwood, Halberstadt, et al). Outside of the body, psilocin is a short-lived and unstable molecule, so use of psilocin must be accomplished by administering psilocybin. However, this is expensive and results in a low yield. Therefore, there is a need to develop new psilocin prodrugs with improved properties for treatment of psychological disorders that are more cost-effective and efficient.

### SUMMARY OF THE DISCLOSURE

The disclosure relates to a compound of formula (I):

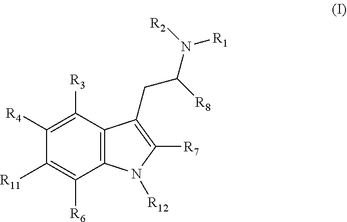

wherein
R₁ is selected from hydrogen, straight chain or branched C₁-C₆ alkyl or a straight chain or branched C₂-C₆ alkenyl;
R₂ is selected from a protecting group, hydrogen, straight chain or branched C₁-C₆ alkyl or a straight chain or branched C₂-C₆ alkenyl;
R₃ and R₄ are independently chosen from hydrogen, hydroxyl, —OR₉, —OC(O)R₅, —OC(O)OR₅, or —OSO₂R₅;
R₅ is a straight chain or branched C₁-C₆ alkyl or a substituted or unsubstituted aryl;
R₉ is selected from a protecting group, a straight chain or branched C₁-C₆ alkyl, or a substituted or unsubstituted aryl;
R₆, R₇, R₈, and R₁₁ are each independently hydrogen or a straight chain or branched C₁-C₆ alkyl; and
R₁₂ is selected from a protecting group, hydrogen, a straight chain or branched C₁-C₆ alkyl or a straight chain or branched C₂-C₆ alkenyl,
wherein at least one of R₂ or R₁₂ is a protecting group;
or a pharmaceutically acceptable acid-addition salt thereof.

The disclosure also relates to a compound of formula (Ia):

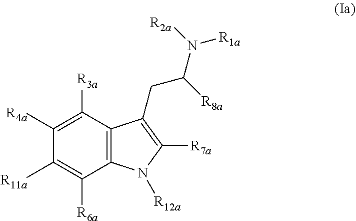

wherein
R₁ₐ is selected from hydrogen, straight chain or branched C₁-C₆ alkyl or a straight chain or branched C₂-C₆ alkenyl;
R₂ₐ is a protecting group;
R₃ₐ and R₄ₐ are independently chosen from hydrogen, hydroxyl, —OR₉ₐ, —OC(O)R₅ₐ, —OC(O)OR₅ₐ, or —OSO₂R₅ₐ;
R₅ₐ is a straight chain or branched C₁-C₆ alkyl or a substituted or unsubstituted aryl;
R₉ₐ is selected from a protecting group, a straight chain or branched C₁-C₆ alkyl, or a substituted or unsubstituted aryl;
R₆ₐ, R₇ₐ, R₈ₐ, and R₁₁ₐ are each independently hydrogen or a straight chain or branched C₁-C₆ alkyl; and
R₁₂ₐ is selected from hydrogen, a straight chain or branched C₁-C₆ alkyl or a straight chain or branched C₂-C₆ alkenyl;
or a pharmaceutically acceptable acid-addition salt thereof.

The disclosure also relates to a compound of formula (Ib):

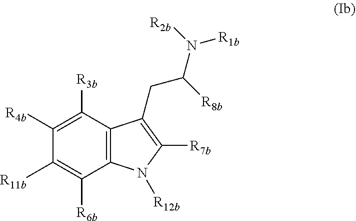

wherein
R_{1b} is selected from hydrogen, straight chain or branched C₁-C₆ alkyl or a straight chain or branched C₂-C₆ alkenyl;
R_{2b} is selected from a protecting group, hydrogen, straight chain or branched C₁-C₆ alkyl or a straight chain or branched C₂-C₆ alkenyl;
R_{3b} and R_{4b} are independently chosen from hydrogen, hydroxyl, —OR_{9b}, —OC(O)R_{5b}, —OC(O)OR_{5b}, or —OSO₂R_{5b};
R_{5b} is a straight chain or branched C₁-C₆ alkyl or a substituted or unsubstituted aryl;
R_{9b} is selected from a protecting group, a straight chain or branched C₁-C₆ alkyl, or a substituted or unsubstituted aryl;
R_{11b} is hydrogen;
R_{12b} is a protecting group; and
R_{6b}, R_{7b}, and R_{8b} are each independently hydrogen or a straight chain or branched C₁-C₆ alkyl;
or a pharmaceutically acceptable acid-addition salt thereof.

The disclosure relates to compositions comprising, consisting essentially of, or consisting of a compound of formula (I), formula (Ia), or formula (Ib) and an excipient. The disclosure also relates pharmaceutical compositions comprising a therapeutically effective amount of a compound of formula (I), formula (Ia), or formula (Ib), wherein the excipient is a pharmaceutically acceptable carrier. The disclosure further relates to a method of preventing or treating a psychological disorder comprising the step of administering to a subject in need thereof a therapeutically effective amount of a compound of formula (I), formula (Ia), or formula (Ib), or of a pharmaceutical composition containing the compound.

The disclosure also relates to a composition comprising, consisting essentially of, or consisting of as a first active component: a compound of formula (I), formula (Ia), or formula (Ib); and as a second active component selected from (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone; and a pharmaceutically acceptable excipient.

The disclosure further relates to methods of preventing or treating a physical and/or psychological disorders comprising the step of administering to a subject in need thereof an effective amount of a compound of formula (I), formula (Ia), or formula (Ib), or a composition (e.g., a pharmaceutically-acceptable composition) comprising a compound of formula (I), formula (Ia), or formula (Ib).

The disclosure also relates to methods of preventing or treating inflammation and/or pain comprising the step of administering to a subject in need thereof a therapeutically effective amount of a compound of formula (I), formula (Ia), or formula (Ib), and to administering a pharmaceutical composition or a composition according to the disclosure.

The disclosure also relates to methods of preventing or treating inflammation and/or pain, preventing or treating a neurological disorder, modulating activity of a mitogen activating protein (MAP), modulating neurogenesis, or modulating neurite outgrowth comprising the step of administering to a subject in need thereof a therapeutically effective amount of a compound of formula (I), formula (Ia), or formula (Ib), and to administering a pharmaceutical composition or a composition according to the disclosure.

The disclosure also relates to methods of preventing or treating sexual health disorders including, but not limited to, hypoactive sexual desire disorder, hyperactive sexual desire disorder, orgasmic disorder, arousal disorder, vaginismus, and dyspareunia. In some embodiments, the disorder is a male sexual dysfunction disorder. In some embodiments. the disorder is a female sexual dysfunction disorder.

The disclosure also relates to methods of preventing or treating women's health disorders including, but not limited to, menstrual cramping, dysmenorrhea, post-hysterectomic pain, vaginal or vulvar vestibule mucosa disorder, vaginal atrophy, or vulvar vestibulitis.

### DETAILED DESCRIPTION

Compounds of the Disclosure

The disclosure relates to a compound of formula (I):

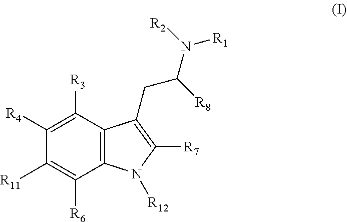

wherein
R₁ is selected from hydrogen, straight chain or branched C₁-C₆ alkyl or a straight chain or branched C₂-C₆ alkenyl;
R₂ is selected from a protecting group, hydrogen, straight chain or branched C₁-C₆ alkyl or a straight chain or branched C₂-C₆ alkenyl;
R₃ and R₄ are independently chosen from hydrogen, hydroxyl, —OR₉, —OC(O)R₅, —OC(O)OR₅, or —OSO₂R₅;
R₅ is a straight chain or branched C₁-C₆ alkyl or a substituted or unsubstituted aryl;
R₉ is selected from a protecting group, a straight chain or branched C₁-C₆ alkyl, or a substituted or unsubstituted aryl;
R₆, R₇, R₈, and R₁₁ are each independently hydrogen or a straight chain or branched C₁-C₆ alkyl; and
R₁₂ is selected from a protecting group, hydrogen, a straight chain or branched C₁-C₆ alkyl or a straight chain or branched C₂-C₆ alkenyl, wherein at least one of R₂ or R₁₂ is a protecting group;
or a pharmaceutically acceptable acid-addition salt thereof.

In formula (I), R₁ is selected from hydrogen, straight chain or branched C₁-C₆ alkyl or a straight chain or branched C₂-C₆ alkenyl. R₁ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁ may be a straight chain or branched C₁-C₄ alkyl, for example a straight chain C₁-C₄ alkyl, or a C₂-C₄ alkenyl. R₁ may be selected from straight chain or branched C₂-C₆ alkyl or C₃-C₆ alkyl. R₁ may be selected from methyl, ethyl, n-propyl, isopropyl, n-butyl, isobutyl, or tert-butyl. In other embodiments, R₁ may be methyl, ethyl, propyl, or isopropyl.

In formula (I), R₁₂ is selected from a protecting group, hydrogen, a straight chain or branched C₁-C₆ alkyl or a straight chain or branched C₂-C₆ alkenyl. R₁₂ may be a protecting group, including where R₁₂ is any acceptable protecting group such as a carbamate. In some embodiments, the protecting group may be selected from —C(O)OR₁₀, wherein R₁₀ is selected from optionally substituted C₁-C₆ alkyl that is branched or unbranched, and optionally substituted aryl. In some embodiments, R₁₂ is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group. R₁₂ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁₂ may be a straight chain or branched C₁-C₄ alkyl, for example a straight chain C₁-C₄ alkyl, or a C₂-C₄ alkenyl. In some embodiments, R₁₂ may be selected from straight chain or branched C₂-C₆ alkyl or C₃-C₆ alkyl. In some embodiments, R₁₂ may be selected from methyl, ethyl, n-propyl, isopropyl, n-butyl, isobutyl, or tert-butyl. In other embodiments, R₁₂ may be methyl, ethyl, propyl, or isopropyl. In some embodiments, R₁₂ may be a straight chain or branched C₂-C₄ alkyl, such as ethyl, n-propyl, isopropyl, n-butyl, sec-butyl, or isobutyl.

In formula (I), R₂ is a protecting group, hydrogen, straight chain or branched C₁-C₆ alkyl or a straight chain or branched C₂-C₆ alkenyl. In formula (I), R₂ is any acceptable protecting group, such as a carbamate. In some embodiments, the protecting group may be selected from —C(O)OR₁₀, wherein R₁₀ is selected from optionally substituted C₁-C₆ alkyl that is branched or unbranched, and optionally substituted aryl. In some embodiments, R₂ is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group. In certain embodiments of formula (I), R₂ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₂ may be a straight chain or branched C₁-C₄ alkyl, for example a straight chain C₁-C₄ alkyl, or a C₂-C₄ alkenyl. R₂ may be selected from straight chain or branched C₂-C₆ alkyl or C₃-C₆ alkyl. R₂ may be selected from methyl, ethyl, n-propyl, isopropyl, n-butyl, isobutyl, or tert-butyl. In other embodiments, R₂ may be methyl, ethyl, propyl, or isopropyl.

In formula (I), R₃ and R₄ are independently chosen from hydrogen, hydroxyl, —OR₉, —OC(O)R₅, —OC(O)OR₅, and —OSO₂R₅. In certain embodiments, at least one of R₃ and R₄ is selected from hydroxyl, —OR₉, —OC(O)R₅, —OC(O)OR₅, and —OSO₂R₅.

In formula (I), R₅ and R₉ are independently for each occurrence selected from straight chain or branched C₁-C₆ alkyl or a substituted or unsubstituted aryl. When R₅ or R₉ is a straight chain or branched C₁-C₆ alkyl, it may be a straight chain or branched C₁-C₄ alkyl, for example a straight chain C₁-C₄ alkyl. R₅ and R₉ may be independently selected from straight chain or branched C₂-C₆ alkyl or C₃-C₆ alkyl. R₅ and R₉ may be independently a methyl, a tert-butyl, a phenyl or a para-tolyl group. In some embodiments, R₅ and R₉ may be independently methyl, ethyl, n-propyl or n-butyl, and for example may be methyl or ethyl R₅ and R₉ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₅ and R₉ may be straight chain or branched C₃-C₅ alkyl. In other embodiments, R₅ and R₉ may be straight chain or branched C₂-C₆ alkyl or C₇-C₁₄ aryl.

In addition to the above, R₉ may also be selected from any acceptable protecting group, such as a carbamate. In some embodiments, the R₉ protecting group may be selected from —C(O)OR₁₀, wherein R₁₀ is selected from optionally substituted C₁-C₆ alkyl that is branched or unbranched, and optionally substituted aryl. In some embodiments, R₉ is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

In some embodiments, R₆, R₇, R₈, and R₁₁ in formula (I) are each independently hydrogen or a straight chain or branched C₁-C₆ alkyl, for example a straight chain C₁-C₆ alkyl. R₆, R₇, R₈, and R₁₁ may be each independently selected from straight chain or branched C₂-C₆ alkyl or C₃-C₆ alkyl. In some embodiments, R₆, R₇, R₈, and R₁₁ may be each independently selected from hydrogen, methyl, ethyl, n-propyl, isopropyl, n-butyl and isobutyl. In other embodiments, R₆, R₇, R₈, and R₁₁ may be independently hydrogen, methyl, or ethyl. In some embodiments, R₇ may be hydrogen or straight chain or branched C₃-C₆ alkyl. In other embodiments R₈ may be hydrogen or straight chain or branched C₂-C₆ alkyl.

In formula (I), at least one of R₂ or R₁₂ is a protecting group.

Pharmaceutically acceptable salts of formula (I) may be any acid (e.g., HX or H₂X) addition salts. The anion, X⁻, may be any pharmaceutically acceptable anion, for example, Cl⁻, I⁻, Br⁻, ascorbate, or hydrofumarate, and the like. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (I) are those with the proviso that R₉ is not methyl when R₄ is —OR₉.

Other exemplary compounds of formula (I) are those with the proviso that when R₃, R₄, R₆, R₇, and R₅ are all hydrogen, R₂ is —C(O)OR₁₀, and R₁ is methyl, then R₁₀ is not benzyl.

Other exemplary compounds of formula (I) are those with the proviso that when R₃ is —OR₉ and R₉ is methyl, then R₂ is neither ethoxycarbonyl or phenoxycarbonyl.

Other exemplary compounds of formula (I) are those with the proviso that when R₄ is —OH then R₁ is a C₁-C₆ alkyl.

Other exemplary compounds of formula (I) include those with the proviso that when R₃, R₆, R₅, and R₁₁ are all hydrogen, R₁ is methyl, R₂ is —C(O)OR₁₀, R₁₂ is methyl, and R₄ is methoxy, then R₁₀ is not ethyl.

Other exemplary compounds of formula (I) include those with the proviso that when R₃, R₆, R₈, and R₁₁ are all hydrogen, R₁ is methyl, R₂ is —C(O)OR₁₀, R₁₂ is hydrogen, and R₄ is methoxy, then R₁₀ is not methyl.

Other exemplary compounds of formula (I) are those with the proviso that R_{3b} is selected from —OR₉, —OC(O)R₅, —OC(O)OR₅, and —OSO₂R₅ when R₁ and R₂ are methyl, R₆, R₇, and R₈ are all hydrogen, and R₁₂ is tert-butyloxycarbonyl (BOC).

Other exemplary compounds of formula (I) are those with the proviso that R₃ is not hydrogen or methoxy when R₁ is hydrogen, R₂ is methyl, R₆, R₇, and R₈ are all hydrogen, and R₁₂ is tert-butyloxycarbonyl (BOC) or methoxycarbonyl.

Other exemplary compounds of formula (I) are those with the proviso that R₃ and R₄ are not both methyl when R₁ and R₂ are methyl, R₆, R₇, and R₈ are all hydrogen, and R₁₂ is ethoxycarbonyl.

This disclosure also relates to tryptamine compounds of formula (Ia):

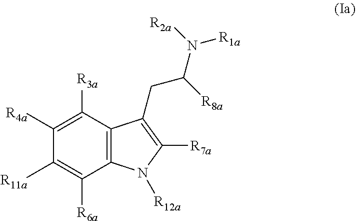

wherein
R₁ₐ is selected from hydrogen, straight chain or branched C₁-C₆ alkyl or a straight chain or branched C₂-C₆ alkenyl;
R₂ₐ is a protecting group;
R₃ₐ and R₄ₐ are independently chosen from hydrogen, hydroxyl, —OR₉ₐ, —OC(O)R₅ₐ, —OC(O)OR₅ₐ, or —OSO₂R₅ₐ;
R₅ₐ is a straight chain or branched C₁-C₆ alkyl or a substituted or unsubstituted aryl;
R₉ₐ is selected from a protecting group, a straight chain or branched C₁-C₆ alkyl, or a substituted or unsubstituted aryl;
R₆ₐ, R₇ₐ, R₈ₐ, and R₁₁ₐ are each independently hydrogen or a straight chain or branched C₁-C₆ alkyl; and
R₁₂ₐ is selected from hydrogen, a straight chain or branched C₁-C₆ alkyl or a straight chain or branched C₂-C₆ alkenyl;
   or a pharmaceutically acceptable acid-addition salt thereof.

In formula (Ia), R₁ₐ is selected from hydrogen, straight chain or branched C₁-C₆ alkyl or a straight chain or branched C₂-C₆ alkenyl. R₁ₐ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁ₐ may be a straight chain or branched C₁-C₄ alkyl, for example a straight chain C₁-C₄ alkyl, or a C₂-C₄ alkenyl. R₁ₐ may be selected from straight chain or branched C₂-C₆ alkyl or C₃-C₆ alkyl. R₁ₐ may be selected from methyl, ethyl, n-propyl, isopropyl, n-butyl, isobutyl, or tert-butyl. In other embodiments, R₁ₐ may be methyl, ethyl, propyl, or isopropyl.

In formula (Ia), R₂ₐ is selected from hydrogen, a straight chain or branched C₁-C₆ alkyl or a straight chain or branched C₂-C₆ alkenyl. R₁₂ₐ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁₂ₐ may be a straight chain or branched C₁-C₄ alkyl, for example a straight chain C₁-C₄ alkyl, or a C₂-C₄ alkenyl. In some embodiments, R₁₂ₐ may be selected from straight chain or branched C₂-C₆ alkyl or C₃-C₆ alkyl. In some embodiments, R₁₂ₐ may be selected from methyl, ethyl, n-propyl, isopropyl, n-butyl, isobutyl, or tert-butyl. In other embodiments, R₁₂ₐ may be methyl, ethyl, propyl, or isopropyl. In some embodiments, R₁₂ₐ may be a straight chain or branched C₂-C₄ alkyl, such as ethyl, n-propyl, isopropyl, n-butyl, sec-butyl, or isobutyl.

In formula (Ia), R₂ is a protecting group. In formula (Ia), R₂ₐ is any acceptable protecting group, such as a carbamate. In some embodiments, the protecting group may be selected from —C(O)OR₁₀ₐ, wherein R₁₀ₐ is selected from optionally substituted C₁-C₆ alkyl that is branched or unbranched, and optionally substituted aryl. In some embodiments, R₂ₐ is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

In formula (Ia), R₃ₐ and R₄ₐ are independently chosen from hydrogen, hydroxyl, —OR₉ₐ, —OC(O)R₅ₐ, —OC(O)OR₅ₐ, and —OSO₂R₅ₐ. In certain embodiments, at least one of R₃ₐ and R₄ₐ is selected from hydroxyl, —OR₉ₐ, —OC(O)R₅ₐ, —OC(O)OR₅ₐ, and —OSO₂R₅ₐ.

In formula (Ia), R₅ₐ and R₉ₐ are independently for each occurrence selected from straight chain or branched C₁-C₆ alkyl or a substituted or unsubstituted aryl. When R₅ₐ or R₉ₐ is a straight chain or branched C₁-C₆ alkyl, it may be a straight chain or branched C₁-C₄ alkyl, for example a straight chain C₁-C₄ alkyl. R₅ₐ and R₉ₐ may be independently selected from straight chain or branched C₂-C₆ alkyl or C₃-C₆ alkyl. R₅ₐ and R₉ₐ may be independently a methyl, a tert-butyl, a phenyl or a para-tolyl group. In some embodiments, R₅ₐ and R₉ₐ may be independently methyl, ethyl, n-propyl or n-butyl, and for example may be methyl or ethyl R₅ₐ and R₉ₐ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₅ₐ and R₉ₐ may be straight chain or branched C₃-C₅ alkyl. In other embodiments, R₅ₐ and R₉ₐ may be straight chain or branched C₂-C₆ alkyl or C₇-C₁₄ aryl.

In addition to the above, R₉ₐ may also be selected from any acceptable protecting group, such as a carbamate. In some embodiments, the R₉ₐ protecting group may be selected from —C(O)OR₁₀ₐ, wherein R₁₀ₐ is selected from optionally substituted C₁-C₆ alkyl that is branched or unbranched, and optionally substituted aryl. In some embodiments, R₉ₐ is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

R₆ₐ, R₇ₐ, R₈ₐ, and R₁₁ₐ in formula (Ia) are each independently hydrogen or a straight chain or branched C₁-C₆ alkyl, for example a straight chain C₁-C₆ alkyl. R₆ₐ, R₇ₐ, R₈ₐ, and R₁₁ₐ may be each independently selected from straight chain or branched C₂-C₆ alkyl or C₃-C₆ alkyl. In some embodiments, R₆ₐ, R₇ₐ, R₈ₐ, and R₁₁ₐ may be each independently selected from hydrogen, methyl, ethyl, n-propyl, isopropyl, n-butyl and isobutyl. In other embodiments, R₆ₐ, R₇ₐ, R₈ₐ, and R₁₁ₐ may be independently hydrogen, methyl, or ethyl. In some embodiments, R₇ₐ may be hydrogen or straight chain or branched C₃-C₆ alkyl. In other embodiments R₅ₐ may be hydrogen or straight chain or branched C₂-C₆ alkyl.

Pharmaceutically acceptable salts of formula (Ia) may be any acid (e.g., HX or H₂X) addition salts. The anion, X⁻, may be any pharmaceutically acceptable anion, for example, Cl⁻, I⁻, Br⁻, ascorbate, or hydrofumarate, and the like. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (Ia) are those wherein R₃ₐ and R₄ₐ are independently hydrogen or straight chain or branched —OR₉ₐ, —OC(O)OR₅ₐ, or —OSO₂R₅ₐ, wherein R₉ₐ is straight chain or branched C₃-C₅ alkyl.

Other exemplary compounds of formula (Ia) are those where one of R₃ₐ and R₄ₐ is hydrogen and the other of R₃ₐ and R₄ₐ is —OC(O)OR₅ₐ or —OSO₂R₅ₐ.

Other exemplary compounds of formula (Ia) are those where R₃ₐ and R₄ₐ are both hydrogen.

Other exemplary compounds of formula (Ia) are those where one of R₃ₐ and R₄ₐ is hydrogen and the other of R₃ₐ and R₄ₐ is —OC(O)OR₅ₐ.

Other exemplary compounds of formula (Ia) are those where R₃ₐ and R₄ₐ are independently selected from hydrogen and —OC(O)R₅ₐ, wherein R₅ₐ is selected from straight chain or branched C₁-C₆ alkyl. Still other exemplary compounds of formula (Ia) are those where one of R₃ₐ and R₄ₐ is hydrogen and the other of R₃ₐ and R₄ₐ is selected from —OC(O)R₅ₐ, wherein R₅ₐ is selected from straight chain or branched C₁-C₆ alkyl.

Other exemplary compounds of formula (Ia) are those wherein at least one of R₃ₐ, R₄ₐ, R₆ₐ, R₇ₐ, and R₈ₐ is not hydrogen.

Other exemplary compounds of formula (Ia) are those with the proviso that R₉ₐ is not methyl when R₄ₐ is —OR₉ₐ.

Other exemplary compounds of formula (Ia) are those wherein R₄ₐ is —OR₉ₐ and R₉ₐ is straight chain or branched C₂-C₆ alkyl.

Other exemplary compounds of formula (Ia) are those wherein one of R₃ₐ and R₄ₐ is hydrogen.

Other exemplary compounds of formula (Ia) are those wherein R₁ₐ is selected from straight chain or branched C₁-C₆ alkyl or a straight chain or branched C₂-C₆ alkenyl.

Other exemplary compounds of formula (Ia) are those with the proviso that when R₃ₐ, R₄ₐ, R₆ₐ, R₇ₐ and R₈ₐ are all hydrogen, R₂ₐ is —C(O)OR₁₀ₐ, and R₁ₐ is methyl, then R₁₀ₐ is not benzyl.

Other exemplary compounds of formula (Ia) are those wherein R₁ₐ is hydrogen.

Other exemplary compounds of formula (Ia) are those with the proviso that when R₃ₐ is —OR₉ₐ and R₉ₐ is methyl, then R₂ₐ is neither ethoxycarbonyl or phenoxycarbonyl.

Other exemplary compounds of formula (Ia) are those with the proviso that when R₄ₐ is —OH then R₁ₐ is a C₁-C₆ alkyl.

Other exemplary compounds of formula (Ia) include those with the proviso that when R₃ₐ, R₆ₐ, R₈ₐ, and R₁₁ₐ are all hydrogen, R₁ₐ is methyl, R₂ₐ is —C(O)OR₁₀ₐ, R₁₂ₐ is methyl, and R₄ₐ is methoxy, then R₁₀ₐ is not ethyl.

Other exemplary compounds of formula (Ia) include those with the proviso that when R₃ₐ, R₆ₐ, R₈ₐ, and R₁₁ₐ are all hydrogen, R₁ₐ is methyl, R₂ₐ is —C(O)OR₁₀ₐ, R₁₂ₐ is hydrogen, and R₄ₐ is methoxy, then R₁₀ₐ is not methyl.

Other exemplary compounds of formula (Ia) are those wherein R₆ₐ is hydrogen.

Other exemplary compounds of formula (Ia) are those wherein R₆ₐ is selected from straight chain or branched C₁-C₆ alkyl.

Other exemplary compounds of formula (Ia) are those wherein R₇ₐ is hydrogen.

Other exemplary compounds of formula (Ia) are those wherein R₇ₐ is selected from straight chain or branched C₁-C₆ alkyl.

Other exemplary compounds of formula (Ia) are those wherein R₈ₐ is hydrogen.

Other exemplary compounds of formula (Ia) are those wherein R₈ₐ is selected from straight chain or branched C₁-C₆ alkyl.

Other exemplary compounds of formula (Ia) are those where R₁ₐ is methyl.

Other exemplary compounds of formula (Ia) are those where R₁ₐ is ethyl.

Other exemplary compounds of formula (Ia) are those where R₁ₐ is straight chain or branched propyl.

Other exemplary compounds of formula (Ia) are those where R₁ₐ is isopropyl.

Other exemplary compounds of formula (Ia) are those where R₁ₐ is straight chain or branched butyl.

Other exemplary compounds of formula (Ia) are those where R₁ₐ is straight chain or branched pentyl.

Other exemplary compounds of formula (Ia) are those where R₁ₐ is straight chain or branched hexyl.

In some embodiments the compound of formula (Ia) is N—BOC-Norpsilocin or a pharmaceutically-acceptable addition salt thereof.

In some embodiments the compound of formula (Ia) is N—BOC-4-Acetoxy-Norpsilocin or a pharmaceutically-acceptable addition salt thereof.

The disclosure also relates to a compound of formula (Ib):

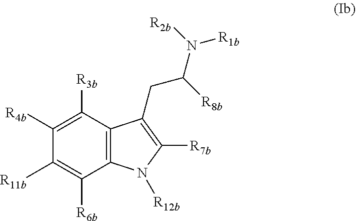

wherein
R_{1b} is selected from hydrogen, straight chain or branched C₁-C₆ alkyl or a straight chain or branched C₂-C₆ alkenyl;
R_{2b} is selected from a protecting group, hydrogen, straight chain or branched C₁-C₆ alkyl or a straight chain or branched C₂-C₆ alkenyl;
R_{3b} and R_{4b} are independently chosen from hydrogen, hydroxyl, —OR_{9b}, —OC(O)R_{5b}, —OC(O)OR_{5b}, or —OSO₂R_{5b};
R_{5b} is a straight chain or branched C₁-C₆ alkyl or a substituted or unsubstituted aryl;
R_{9b} is selected from a protecting group, a straight chain or branched C₁-C₆ alkyl, or a substituted or unsubstituted aryl;
R_{11b} is hydrogen;
R_{12b} is a protecting group; and
R_{6b}, R_{7b}, and R_{8b} are each independently hydrogen or a straight chain or branched C₁-C₆ alkyl;
   or a pharmaceutically acceptable acid-addition salt thereof.

In formula (Ib), R_{1b} is selected from hydrogen, straight chain or branched C₁-C₆ alkyl or a straight chain or branched C₂-C₆ alkenyl. R_{1b} may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R_{1b} may be a straight chain or branched C₁-C₄ alkyl, for example a straight chain C₁-C₄ alkyl, or a C₂-C₄ alkenyl. R_{1b} may be selected from straight chain or branched C₂-C₆ alkyl or C₃-C₆ alkyl. R_{1b} may be selected from methyl, ethyl, n-propyl, isopropyl, n-butyl, isobutyl, or tert-butyl. In other embodiments, R_{1b} may be methyl, ethyl, propyl, or isopropyl.

In formula (Ib), R_{2b} is selected from a protecting group, hydrogen, straight chain or branched C₁-C₆ alkyl or a straight chain or branched C₂-C₆ alkenyl. In certain embodiments of formula (Ib), R_{2b} may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R_{2b} may be a straight chain or branched C₁-C₄ alkyl, for example a straight chain C₁-C₄ alkyl, or a C₂-C₄ alkenyl. R_{2b} may be selected from straight chain or branched C₂-C₆ alkyl or C₃-C₆ alkyl. R_{2b} may be selected from methyl, ethyl, n-propyl, isopropyl, n-butyl, isobutyl, or tert-butyl. In other embodiments, R_{2b} may be methyl, ethyl, propyl, or isopropyl. In certain embodiments of formula (Ib), R_{2b} is any acceptable protecting group, such as a carbamate. In some embodiments, the protecting group may be selected from —C(O)OR_{10b}, wherein R_{10b} is selected from optionally substituted C₁-C₆ alkyl that is branched or unbranched, and optionally substituted aryl. In some embodiments, R_{2b} is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

In formula (Ib), R_{3b} and R_{4b} are independently chosen from hydrogen, hydroxyl, —OR_{9b}, —OC(O)R_{5b}, —OC(O)OR_{5b}, and —OSO₂R_{5b}.

In formula (Ib), R_{5b} and R_{9b} are independently for each occurrence selected from straight chain or branched C₁-C₆ alkyl or a substituted or unsubstituted aryl. When R_{5b} or R_{9b} is a straight chain or branched C₁-C₆ alkyl, it may be a straight chain or branched C₁-C₄ alkyl, for example a straight chain C₁-C₄ alkyl. R_{5b} and R_{9b} may be independently selected from straight chain or branched C₂-C₆ alkyl or C₃-C₆ alkyl. R_{5b} and R_{9b} may be independently a methyl, a tert-butyl, a phenyl or a para-tolyl group. In some embodiments, R₅ and R_{9b} may be independently methyl, ethyl, n-propyl or n-butyl, and for example may be methyl or ethyl R_{5b} and R_{9b} may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R_{5b} and R_{9b} may be straight chain or branched C₃-C₅ alkyl. In other embodiments, R_{5b} and R_{9b} may be straight chain or branched C₂-C₆ alkyl or C₇-C₁₄ aryl.

In addition to the above, R_{9b} may also be selected from any acceptable protecting group, such as a carbamate. In some embodiments, the R_{9b} protecting group may be selected from —C(O)OR_{10b}, wherein R_{10b} is selected from optionally substituted C₁-C₆ alkyl that is branched or unbranched, and optionally substituted aryl. In some embodiments, R_{9b} is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

R_{6b}, R_{7b}, and R_{8b} in formula (Ib) are each independently hydrogen or a straight chain or branched C₁-C₆ alkyl, for example a straight chain C₁-C₆ alkyl. R_{6b}, R_{7b}, and R_{8b} may be each independently selected from straight chain or branched C₂-C₆ alkyl or C₃-C₆ alkyl. In some embodiments, R_{6b}, R_{7b}, and R_{8b} may be each independently selected from hydrogen, methyl, ethyl, n-propyl, isopropyl, n-butyl and isobutyl. In other embodiments, R_{6b}, R_{7b}, and R_{8b} may be independently hydrogen, methyl, or ethyl. In some embodiments, R_{7b} may be hydrogen or straight chain or branched C₃-C₆ alkyl. In other embodiments R_{8b} may be hydrogen or straight chain or branched C₂-C₆ alkyl.

In formula (Ib), R_{11b} is hydrogen.

R_{12b} in formula (Ib) is a protecting group, including where R₁₂ is any acceptable protecting group, such as a carbamate. In some embodiments, the protecting group may be selected from —C(O)OR_{10b}, wherein R_{10b} is selected from optionally substituted C₁-C₆ alkyl that is branched or unbranched, and optionally substituted aryl. In some embodiments, R_{12b} is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

Pharmaceutically acceptable salts of formula (Ib) may be any acid (e.g., HX or H₂X) addition salts. The anion, X⁻, may be any pharmaceutically acceptable anion, for example, Cl⁻, I⁻, Br⁻, ascorbate, or hydrofumarate, and the like. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (Ib) are those wherein R_{3b} and R_{4b} are independently hydrogen or straight chain or branched —OR_{9b}, —OC(O)OR_{5b}, or —OSO₂R_{5b}, wherein R_{9b} is straight chain or branched C₃-C₅ alkyl.

Other exemplary compounds of formula (Ib) are those where at least one of R_{3b}, R_{4b}, R_{6b}, R_{7b}, and R_{8b} is not hydrogen.

Other exemplary compounds of formula (Ib) are those where at least one of R_{3b} or R_{4b} is not methyl.

Other exemplary compounds of formula (Ib) are those where R_{1b} is hydrogen, R_{2b} is methyl, and at least one of R_{3b} or R_{4b} is not hydrogen.

Other exemplary compounds of formula (Ib) are those where one of R_{3b} and R_{4b} is hydrogen and the other of R_{3b} and R_{4b} is —OC(O)OR_{5b} or —OSO₂R_{5b}.

Other exemplary compounds of formula (Ib) are those where R_{3b} and R_{4b} are both hydrogen.

Other exemplary compounds of formula (Ib) are those where one of R_{3b} and R_{4b} is hydrogen and the other of R_{3b} and R_{4b} is —OC(O)OR_{5b}.

Other exemplary compounds of formula (Ib) are those where R_{3b} and R_{4b} are independently selected from hydrogen and —OC(O)R_{5b}, wherein R_{5b} is selected from straight chain or branched C₁-C₆ alkyl.

Still other exemplary compounds of formula (Ib) are those where one of R_{3b} and R_{4b} is hydrogen and the other of R_{3b} and R_{4b} is selected from —OC(O)R_{5b}, wherein R_{5b} is selected from straight chain or branched C₁-C₆ alkyl.

Other exemplary compounds of formula (Ib) are those wherein at least one of R_{3b}, R_{4b}, R_{6b}, R_{7b}, and R_{8b} is not hydrogen.

Other exemplary compounds of formula (Ib) are those with the proviso that R_{9b} is not methyl when R_{4b} is —OR_{9b}.

Other exemplary compounds of formula (Ib) are those wherein R_{4b} is —OR_{9b} and R_{9b} is straight chain or branched C₂-C₆ alkyl.

Other exemplary compounds of formula (Ib) are those wherein one of R_{3b} and R_{4b} is hydrogen.

Other exemplary compounds of formula (Ib) are those wherein R_{1b} is selected from straight chain or branched C₁-C₆ alkyl or a straight chain or branched C₂-C₆ alkenyl.

Other exemplary compounds of formula (Ib) are those with the proviso that when R_{3b}, R_{4b}, R_{6b}, R_{7b}, and R_{8b} are all hydrogen, R_{2b} is —C(O)OR_{10b}, and R_{1b} is methyl, R_{10b} is not benzyl.

Other exemplary compounds of formula (Ib) are those wherein R_{1b} is hydrogen.

Other exemplary compounds of formula (Ib) are those with the proviso that when R_{4b} is —OH then R_{1b} is a C₁-C₆ alkyl.

Other exemplary compounds of formula (Ib) are those wherein R_{6b} is hydrogen.

Other exemplary compounds of formula (Ib) are those wherein R_{6b} is selected from straight chain or branched C₁-C₆ alkyl.

Other exemplary compounds of formula (Ib) are those wherein R_{7b} is hydrogen.

Other exemplary compounds of formula (Ib) are those wherein R_{7b} is selected from straight chain or branched C₁-C₆ alkyl.

Other exemplary compounds of formula (Ib) are those wherein R_{8b} is hydrogen.

Other exemplary compounds of formula (Ib) are those wherein R_{8b} is selected from straight chain or branched C₁-C₆ alkyl.

Other exemplary compounds of formula (Ib) are those where R_{1b} is hydrogen.

Other exemplary compounds of formula (Ib) are those where R_{1b} is methyl.

Other exemplary compounds of formula (Ib) are those where R_{1b} is ethyl.

Other exemplary compounds of formula (Ib) are those where R_{1b} is straight chain or branched propyl.

Other exemplary compounds of formula (Ib) are those where R_{1b} is isopropyl.

Other exemplary compounds of formula (Ib) are those where R_{1b} is straight chain or branched butyl.

Other exemplary compounds of formula (Ib) are those where R_{1b} is straight chain or branched pentyl.

Other exemplary compounds of formula (Ib) are those where R_{1b} is straight chain or branched hexyl.

Other exemplary compounds of formula (Ib) are those where R_{2b} is hydrogen.

Other exemplary compounds of formula (Ib) are those where R_{2b} is methyl.

Other exemplary compounds of formula (Ib) are those where R_{2b} is ethyl.

Other exemplary compounds of formula (Ib) are those where R_{2b} is straight chain or branched propyl.

Other exemplary compounds of formula (Ib) are those where R_{2b} is isopropyl.

Other exemplary compounds of formula (Ib) are those where R_{2b} is straight chain or branched butyl.

Other exemplary compounds of formula (Ib) are those where R_{2b} is straight chain or branched pentyl.

Other exemplary compounds of formula (Ib) are those where R_{2b} is straight chain or branched hexyl.

Other exemplary compounds of formula (Ib) are those where R_{12b} is —C(O)OR_{10b}, and wherein R_{10b} is selected from optionally substituted C₁-C₆ alkyl that is branched or unbranched, and optionally substituted aryl.

Other exemplary compounds of formula (Ib) are those where R_{12b} is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

Other exemplary compounds of formula (Ib) are those with the proviso that R_{3b} is selected from —OR_{9b}, —OC(O)R_{5b}, —OC(O)OR_{5b}, and —OSO₂R_{5b} when R_{1b} and R_{2b} are methyl, R_{6b}, R_{7b}, and R_{8b} are all hydrogen, and R_{12b} is tert-butyloxycarbonyl (BOC).

Other exemplary compounds of formula (Ib) are those with the proviso that R_{3b} is not hydrogen or methoxy when R_{1b} is hydrogen, R_{2b} is methyl, R_{6b}, R_{7b}, and R_{8b} are all hydrogen, and R_{12b} is tert-butyloxycarbonyl (BOC) or methoxycarbonyl.

Other exemplary compounds of formula (Ib) are those with the proviso that R_{3b} and R_{4b} are not both methyl when R_{1b} and R_{2b} are methyl, R_{6b}, R_{7b}, and R_{8b} are all hydrogen, and R_{12b} is ethoxycarbonyl.

Other exemplary compounds of formula (Ib) are those wherein R_{1b} is methyl; R_{2b} is selected from hydrogen, methyl, and a protecting group; R_{3b} is selected from hydroxyl, —OR_{9b}, —OC(O)R_{5b}, —OC(O)OR_{5b}, or —OSO₂R_{5b}; and R_{4b}, R_{6b}, R_{7b}, and R_{8b} are all hydrogen. In certain embodiments, R_{2b} is hydrogen. In certain embodiments, R_{3b} is hydroxyl or —OC(O)R_{5b}.

The disclosure also relates to purified tryptamine compounds of formula (I):

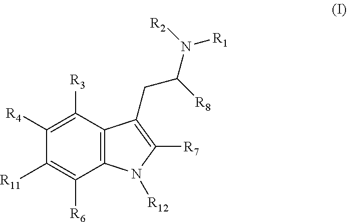

wherein
R₁ is selected from hydrogen, straight chain or branched C₁-C₆ alkyl or a straight chain or branched C₂-C₆ alkenyl;
R₂ is selected from a protecting group, hydrogen, straight chain or branched C₁-C₆ alkyl or a straight chain or branched C₂-C₆ alkenyl;
R₃ and R₄ are independently chosen from hydrogen, hydroxyl, —OR₉, —OC(O)R₅, —OC(O)OR₅, or —OSO₂R₅;
R₅ is a straight chain or branched C₁-C₆ alkyl or a substituted or unsubstituted aryl;
R₉ is selected from a protecting group, a straight chain or branched C₁-C₆ alkyl, or a substituted or unsubstituted aryl;
R₆, R₇, R₈, and R₁₁ are each independently hydrogen or a straight chain or branched C₁-C₆ alkyl; and
R₁₂ is selected from a protecting group, hydrogen, a straight chain or branched C₁-C₆ alkyl or a straight chain or branched C₂-C₆ alkenyl,
wherein at least one of R₂ or R₁₂ is a protecting group;
or a pharmaceutically acceptable acid-addition salt thereof;
   wherein the purity of the tryptamine compound of formula (I) is greater than 95%, greater than 98%, greater than 99%, or greater than 99.9%.

### Methods of Treatment and Therapeutic Uses

Compounds of formula (I), formula (Ia), or formula (Ib) according to the disclosure, crystalline forms thereof, and the methods and the compositions (e.g., pharmaceutical compositions) are used to regulate the activity of a neurotransmitter receptor by administering a therapeutically effective dose of compounds of formula (I), formula (Ia), or formula (Ib) according to the disclosure, and the methods and the compositions (e.g., pharmaceutical compositions) are used to treat inflammation and/or pain by administering a therapeutically effective dose of compounds of formula (I), formula (Ia), or formula (Ib) according to the disclosure.

Methods of the disclosure also related to the administration of a therapeutically effective amount of compounds of formula (I), formula (Ia), or formula (Ib) according to the disclosure to prevent or treat a disease or condition, such as those discussed below for a subject in need of treatment. Compounds of formula (I), formula (Ia), or formula (Ib) according to the disclosure may be administered neat or as a composition comprising compounds of formula (I), formula (Ia), or formula (Ib) according to the disclosure as discussed below.

Compounds of formula (I), formula (Ia), or formula (Ib) according to the disclosure may be used to prevent and/or treat a psychological disorder. The disclosure provides a method for preventing and/or treating a psychological disorder by administering to a subject in need thereof a therapeutically effective amount of compounds of formula (I), formula (Ia), or formula (Ib) according to the disclosure, including the exemplary embodiments discussed herein. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

Compounds of formula (I), formula (Ia), or formula (Ib) according to the disclosure may be used to prevent and/or treat a brain disorder. The disclosure provides a method for preventing and/or treating a brain disorder (e.g., Huntington's disease, Alzheimer's disease, dementia, and Parkinson's disease) by administering to a subject in need thereof a therapeutically effective amount of compounds of formula (I), formula (Ia), or formula (Ib) according to the disclosure, including the exemplary embodiments discussed above.

Compounds of formula (I), formula (Ia), or formula (Ib) according to the disclosure may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of compounds of formula (I), formula (Ia), or formula (Ib) according to the disclosure, including the exemplary embodiments discussed above.

Compounds of formula (I), formula (Ia), or formula (Ib) according to the disclosure may be used to prevent and/or treat inflammation and/or pain, such as for example inflammation and/or pain associated with inflammatory skeletal or muscular diseases or conditions. The disclosure provides a method for preventing and/or treating an inflammation and/or pain by administering to a subject in need thereof a therapeutically effective amount of compounds of formula (I), formula (Ia), or formula (Ib) according to the disclosure, including the exemplary embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibular joint syndrome, and fibromyalgia.

Compounds of formula (I), formula (Ia), or formula (Ib) according to the disclosure may be used to modulate activity of a mitogen activating protein (MAP), comprising administering a composition of the disclosure. In one embodiment, the mitogen activating protein (MAP) comprises a MAP kinase (MAPk). MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, Janus Kinase 1 (JAK1), and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-α. In the central nervous system, p38-alpha regulates neuronal death and neurite degeneration, and it is a common target of Alzheimer's disease therapies. JAK1 influences cytokine signaling, including IL-2, IL-4, IFN-alpha/beta, IFN-γ, and IL-10, and it is implicated in brain aging. JNK3 is neuronal specific protein isoform of the JNKs. It is involved with the regulation of apoptosis. JNK3 also plays a role in modulating the response of cytokines, growth factors, and oxidative stress.

As used herein, the term “modulating activity of a mitogen activating protein” refers to changing, manipulating, and/or adjusting the activity of a mitogen activating protein. In one embodiment, modulating the activity of a MAP, such as a MAPK, can influence neural health, neurogenesis, neural growth and differentiation, and neurodegenerative diseases.

Compounds of formula (I), formula (Ia), or formula (Ib) according to the disclosure may be used to modulate neurogenesis, comprising administering a composition of the disclosure. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

Compounds of formula (I), formula (Ia), or formula (Ib) according to the disclosure may be used to modulate neurite outgrowth, comprising administering a composition of the disclosure. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

The disclosure also relates to methods of preventing or treating sexual health disorders including, but not limited to, hypoactive sexual desire disorder, hyperactive sexual desire disorder, orgasmic disorder, arousal disorder, vaginismus, and dyspareunia. In some embodiments, the disorder is a male sexual dysfunction disorder. In some embodiments. the disorder is a female sexual dysfunction disorder.

The disclosure also relates to methods of preventing or treating women's health disorders including, but not limited to, menstrual cramping, dysmenorrhea, post-hysterectomic pain, vaginal or vulvar vestibule mucosa disorder, menopausal-related disorders, vaginal atrophy, or vulvar vestibulitis.

Compounds of formula (I), formula (Ia), or formula (Ib) according to the disclosure may be used to generate tryptamine compounds. In some embodiments, this can be accomplished by exposing compounds of formula (I), formula (Ia), or formula (Ib) to conditions under which at least one of the protecting groups R₂, R₂ₐ, R_{2b}, R₉, or R₁₂ is cleaved to liberate a compound of formula (I), formula (Ia), or formula (Ib) in which at least one of R₂, R₂ₐ, R_{2b}, R₉, or R₁₂ is converted to a hydrogen atom. Thus, in certain embodiments, cleavage of the R₂, R₂ₐ, or R_{2b} protecting group results in a compound of formula (I), formula (Ia), or formula (Ib) that is a primary tryptamine compound (i.e., wherein R₁ and R₂ are hydrogen, wherein R₁ₐ and R₂ₐ are hydrogen, and wherein R_{1b} and R_{2b} are hydrogen) when R₁, R₁ₐ, or Rib is initially a hydrogen atom. In certain embodiments, cleavage of the R₂, R₂ₐ, or R_{2b} protecting group results in a monoalkyltryptamine compound (i.e., wherein R₂, R₂ₐ, or R_{2b} is hydrogen and R₁, R₁ₐ, or R_{1b} is alkyl or alkenyl) when R₁, R₁ₐ, or R_{1b} is initially selected from a straight chain or branched C₁-C₆ alkyl or a straight chain or branched C₂-C₆ alkenyl.

In certain embodiments, a method of generating a tryptamine compound in situ in a patient is described, the method comprising administering to the patient a compound according to formula (I), formula (Ia), or formula (Ib). In certain embodiments, a method of generating a tryptamine compound is described, the method comprising contacting at least one compound according to formula (I), formula (Ia), or formula (Ib) with an enzyme capable of removing at least one protecting group of R₂, R₂ₐ, R_{2b}, R₉, or R₁₂. In certain embodiments, the enzyme comprises a CYP enzyme. In certain embodiments, the enzyme is provided in an in vitro assay. In certain embodiments, the CYP enzyme is endogenously provided by a patient.

Compositions

The disclosure also relates to compositions comprising an effective amount of a compound of formula (I), formula (Ia), or formula (Ib) according to the disclosure (protected tryptamine compounds of the disclosure), including its exemplary embodiments discussed above, and an excipient (e.g., a pharmaceutically-acceptable excipient). In another embodiment, the disclosure also relates to pharmaceutical compositions comprising a therapeutically effective amount of protected tryptamine compounds of the disclosure, including their exemplary embodiments discussed above, and a pharmaceutically acceptable excipient (also known as a pharmaceutically acceptable carrier). As discussed above, a protected tryptamine compound of the disclosure may be, for example, therapeutically useful to prevent and/or treat the psychological disorders, brain disorders, pain, and inflammation as well as the other disorders described herein.

A composition or a pharmaceutical composition of the disclosure may be in any form which contains a protected tryptamine compound of the disclosure. The composition may be, for example, a tablet, capsule, liquid suspension, injectable, topical, or transdermal. The compositions generally contain, for example, about 1% to about 99% by weight of a protected tryptamine compound of the disclosure and, for example, 99% to 1% by weight of at least one suitable pharmaceutically acceptable excipient. In one embodiment, the composition may be between about 5% and about 75% by weight of a protected tryptamine compound of the disclosure, with the rest being at least one suitable pharmaceutically acceptable excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851 A1 are incorporated herein by reference. According to this disclosure, a protected tryptamine compound of the disclosure may be used as the “first purified psilocybin derivative” in the compositions described in US 2018/0221396 A1 and US 2019/0142851 A1. Accordingly, this disclosure provides a composition comprising: a first component comprising at least one protected tryptamine compound of the disclosure; at least one second component selected from at least one of (a) a serotonergic drug, (b) a purified psilocybin derivative, (c) a purified cannabinoid or (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising at least one protected tryptamine compound of the disclosure with a second component selected from at least one of (a) a serotonergic drug, (b) a purified psilocybin derivative, (c) a purified cannabinoid, or (d) a purified terpene, the compositions represent particular embodiments of the disclosure. Compositions having as a first component at least one protected tryptamine compound of the disclosure with a second component selected from at least one of (e) an adrenergic drug, (f) a dopaminergic drug, (g) a monoamine oxidase inhibitor, (h) a purified erinacine, or (i) a purified hericenone, also represent additional particular embodiments of the disclosure represented by the compositions having the protected tryptamine compound of the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments. Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) at least one protected tryptamine compound of the disclosure and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and (c) a pharmaceutically acceptable excipient. In some embodiments, the protected tryptamine compound(s) of the disclosure and the second active compound(s) are each present in a therapeutically effective amount using a purposefully engineered and unnaturally occurring molar ratios. Exemplary molar ratios of the protected tryptamine compounds of the disclosure to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing a protected tryptamine compound of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. The disclosures of US 2018/0221396 A1 and US 2019/0142851 A1 are incorporated herein by reference. According to this disclosure composition containing a protected tryptamine compound of the disclosure may be used in place of a “purified psilocybin derivative” in the compositions described in US 2018/0221396 A1 and US 2019/0142851 A1.

Accordingly, the disclosure provides a pharmaceutical formulation comprising as (a) at least one protected tryptamine compound of the disclosure and at least one second component selected from (b) a purified psilocybin derivative, (c) a purified cannabinoid or (d) a purified terpene; and at least one pharmaceutically-acceptable excipient or at least one other adjuvant, as described herein. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Some exemplary serotonergic drugs include SSRIs and SNRIs. Some examples of specific serotonergic drugs include the following molecules, including any salts, solvates, or polymorphs thereof: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl]dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (A8-THC), Delta-8-tetrahydrocannabinolic acid (A8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, or xylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In on embodiment, a MAOI inhibits the activity of one or both monoamine oxidase A and monoamine oxidase B. In one embodiment a MAOI is a reversible inhibitors of monoamine oxidase A. In one embodiment a MAOI is a drug chosen from isocarboxazid, phenelzine, or tranylcypromine.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D. In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of a protected tryptamine compounds of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone in exemplary molar ratios are shown in Table 1. A protected tryptamine compound of the disclosure may be any one of the exemplary embodiments described above including their crystalline forms as disclosed herein.

TABLE 1

| | Molar ratio of a | Molar ratio of a | Molar ratio of a |
|---|---|---|---|
| | protected | protected | protected |
| | tryptamine | tryptamine | tryptamine |
| | compound:second | compound:second | compound:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

Exemplary pharmaceutical compositions of a protected tryptamine compound of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, and a purified hericenone and an excipient with exemplary molar ratios of a protected tryptamine compound to the second compound are shown in Table 2. A protected tryptamine compound of the disclosure may be any one of the exemplary embodiments described above including their crystalline forms as disclosed herein.

TABLE 2

| | Molar ratio of a | Molar ratio of a | Molar ratio of a |
|---|---|---|---|
| | protected | protected | protected |
| | tryptamine | tryptamine | tryptamine |
| | compound:second | compound:second | compound:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

An “effective amount” or a “therapeutically effective amount” of a protected tryptamine compound of the disclosure is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. A protected tryptamine compound of the disclosure and pharmaceutical compositions containing it may be used in combination with other agents that are generally administered to a patient being treated for psychological and other disorders discussed above. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. Exemplary carriers include those that do not substantially alter the structure or activity of protected tryptamine compound of the disclosure, nor produce undesirable biological effects or otherwise interact in a deleterious manner with any other component(s) of the pharmaceutical composition.

The pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, a 4-HO-DPT compound of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alignates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like, (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. In some embodiments, the excipient is not water. In some embodiments, the excipient is not a solvent (e.g., EtOH, diethyl ether, ethyl acetate, or hydrocarbon-based solvents (e.g., hexanes). In some embodiments, the dosage form is substantially free of water and/or solvents, for example less than about 5% water by mass, less than 2% water by mass, less than 1% water by mass, less than 0.5% water by mass, or less than 0.1% water by mass.

Excipients or pharmaceutically acceptable adjuvants known in the pharmaceutical formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of protected tryptamine compounds of the disclosure in pure form or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### EXAMPLES

### Example 1

Synthesis of N-Boc-Norpsilocin

3-(2-(methylamino)ethyl)-1H-indol-4-ol (0.125 g, 0.65 mmol, 1 equiv) was added to a dry reaction vial containing anhydrous dichloromethane (DCM) (8 mL) under nitrogen and the contents were cooled to 0° C. under an ice-bath. Triethyl amine (1.2 equiv) was added to the resulting solution at 0° C. followed by Boc-anhydride (1.1 equiv) in a dropwise manner and the contents were then stirred at room temperature until the disappearance of the starting material (per TLC). After the reaction was completed, the contents were diluted with 15 mL DCM and washed twice with cold water followed by brine. The organic layer was then dried under sodium sulfate and reduced under pressure to yield N-Boc-Norpsilocin.

### Example 2

Synthesis of N-Boc-4-OAc-Norpsilocin

Anhydrous DCM (10 mL) was added under nitrogen to a dry reaction vial containing the N-Boc-Norpsilocin obtained in Example 1, used without further purification, and the contents were cooled to 0° C. under an ice-bath. Triethyl amine (2 equiv) was added to the resulting solution at 0° C. followed by acetyl chloride (1.5 equiv) in a dropwise manner and the contents were then stirred at room temperature until the disappearance of the starting material (per TLC). After the reaction completed, the contents were diluted with 20 mL DCM and washed thrice with cold water followed by brine. The organic layer was then dried under sodium sulfate and reduced under pressure to yield a semi solid residue of N-Boc-4-OAc-Norpsilocin.

### REFERENCES

Carhart-Harris, R. L. & Goodwin, G. M. (2017). Neuropsychopharmacology, 42, 2105-2113.
Dinis-Oliveira, R. J. (2017). Drug Metab. Rev. 49, 84-91.
Johnson, M. W. & Griffiths, R. R. (2017). Neurotherapeutics 14, 734-740.
A. M. Sherwood, A. L. Halberstadt, A. K. Klein, J. D. McCorvy, K. W. Kaylo, R. B. Kargbo and P. Meisenheimer, J. Nat. Prod., Article ASAP, DOI: 10.1021/acs.jnatprod.9b01061.

1-54. (canceled)

55. A tryptamine compound selected from the group consisting of:
a tryptamine compound of formula (Ia): 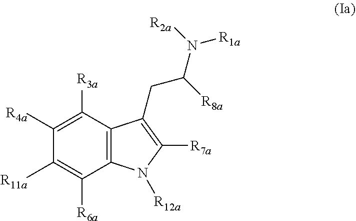
wherein
R₁ₐ is selected from a straight chain or branched C₁-C₆ alkyl or a straight chain or branched C₂-C₆ alkenyl;
R₂ₐ is —C(O)OR₁₀ₐ, and wherein R₁₀ₐ is selected from optionally substituted C₁-C₆ alkyl that is branched or unbranched, and optionally substituted aryl;
R₃ₐ and R₄ₐ are independently chosen from hydrogen, hydroxyl, —OR₉ₐ, —OC(O)R₅ₐ, —OC(O)OR₅ₐ, or —OSO₂R₅ₐ, provided that at least one of R₃ₐ or R₄ₐ is hydroxyl, —OR₉ₐ, —OC(O)R₅ₐ, —OC(O)OR₅ₐ, or —OSO₂R₅ₐ;
R₅ₐ is a straight chain or branched C₁-C₆ alkyl or a substituted or unsubstituted aryl;
R₉ₐ is selected from a protecting group, a straight chain or branched C₁-C₆ alkyl, or a substituted or unsubstituted aryl; and
R₆ₐ, R₇ₐ, R₈ₐ, and R₁₁ₐ are each independently hydrogen or a straight chain or branched C₁-C₆ alkyl; and
R₁₂ₐ is selected from hydrogen, a straight chain or branched C₁-C₆ alkyl or a straight chain or branched C₂-C₆ alkenyl;
or a pharmaceutically acceptable acid-addition salt thereof;
with the proviso that when R₃ₐ is —OR₉ₐ and R₉ₐ is methyl, then R₂ₐ is neither ethoxycarbonyl or phenoxycarbonyl;
with the proviso that when R₃ₐ, R₆ₐ, R₈ₐ, and R₁₁ₐ are all hydrogen, R₁ₐ is methyl, R₂ₐ is —C(O)OR₁₀ₐ, R₁₂ₐ is methyl, and R₄ₐ is methoxy, then R₁₀ₐ is not ethyl; and
with the proviso that when R₃ₐ, R₆ₐ, R₈ₐ, and R₁₁ₐ are all hydrogen, R₁ₐ is methyl, R₂ₐ is —C(O)OR₁₀ₐ, R₁₂ₐ is hydrogen, and R₄ₐ is methoxy, then R₁₀ₐ is not methyl;
a tryptamine compound of formula (Ib): 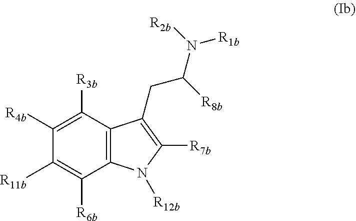
wherein:
R_{1b} is selected from hydrogen, straight chain or branched C₁-C₆ alkyl or a straight chain or branched C₂-C₆ alkenyl;
R_{2b} is selected from a protecting group, hydrogen, straight chain or branched C₁-C₆ alkyl or a straight chain or branched C₂-C₆ alkenyl;
R_{3b} and R_{4b} are independently chosen from hydrogen, hydroxyl, —OR_{9b}, —OC(O)R_{5b}, —OC(O)OR_{5b}, or —OSO₂R_{5b}, provided that at least one of R_{3b} or R_{4b} is hydroxyl, —OR_{9b}, —OC(O)R_{5b}, —OC(O)OR_{5b}, or —OSO₂R_{5b};
R_{5b} is a straight chain or branched C₁-C₆ alkyl or a substituted or unsubstituted aryl;
R_{9b} is selected from a protecting group, a straight chain or branched C₁-C₆ alkyl, or a substituted or unsubstituted aryl;
R_{11b} is hydrogen;
R_{12b} is a protecting group; and
R_{6b}, R_{7b}, and R_{8b} are each independently hydrogen or a straight chain or branched C₁-C₆ alkyl;
or a pharmaceutically acceptable acid-addition salt thereof;
with the proviso that when R_{1b} is hydrogen, R_{2b} is methyl, R_{6b}, R_{7b}, and R_{8b} are all hydrogen, and R_{12b} is tert-butyloxycarbonyl (BOC) or methoxycarbonyl, then R_{3b} is not hydrogen or methoxy; and
with the proviso that when R_{1b} and R_{2b} are methyl, R_{6b}, R_{7b}, and R_{8b} are all hydrogen, and R_{12b} is ethoxycarbonyl, then R_{3b} and R_{4b} are not both methyl; and
a tryptamine compound of formula (I): 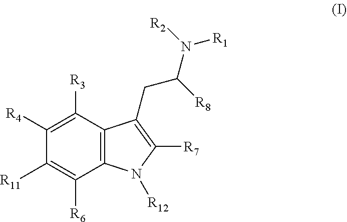
wherein
R₁ is selected from hydrogen, straight chain or branched C₁-C₆ alkyl or a straight chain or branched C₂-C₆ alkenyl;
R₂ is selected from a protecting group, hydrogen, straight chain or branched C₁-C₆ alkyl or a straight chain or branched C₂-C₆ alkenyl;
R₃ and R₄ are independently chosen from hydrogen, hydroxyl, —OR₉, —OC(O)R₅, —OC(O)OR₅, or —OSO₂R₅;
R₅ is a straight chain or branched C₁-C₆ alkyl or a substituted or unsubstituted aryl;
R₉ is selected from a protecting group, a straight chain or branched C₁-C₆ alkyl, or a substituted or unsubstituted aryl;
R₆, R₇, R₈, and R₁₁ are each independently hydrogen or a straight chain or branched C₁-C₆ alkyl; and
R₁₂ is selected from a protecting group, hydrogen, a straight chain or branched C₁-C₆ alkyl or a straight chain or branched C₂-C₆ alkenyl,
wherein at least one of R₂ or R₁₂ is a protecting group;
or a pharmaceutically acceptable acid-addition salt thereof;
wherein the purity of the tryptamine compound of formula (I) is greater than 98%.

56. The compound of claim 55, wherein the tryptamine compound is a compound of formula (Ia).

57. The compound of claim 56, wherein R₂ₐ and R₉ₐ are independently selected from a tert-butyloxycarbonyl (BOC) group, a fluorenylmethyloxycarbonyl (FMOC) group, a benzyloxycarbonyl (Cbz or Z) group, an allyloxycarbonyl (Alloc) group, a 2,2,2-Trichloroethoxycarbonyl (Troc) group, or a 2-(Trimethylsilyl)ethoxycarbonyl (Teoc) group.

58. The compound of claim 56, wherein at least one of R₃ₐ, R₄ₐ, R₆ₐ, R₇ₐ, and R₈ₐ is not hydrogen.

59. The compound of claim 56, with the proviso that R₉ₐ is not methyl when R₄ₐ is —OR₉ₐ.

60. The compound of claim 55, wherein the tryptamine compound is a compound of formula (Ib).

61. The compound of claim 60, wherein R_{12b} is selected from a tert-butyloxycarbonyl (BOC) group, a fluorenylmethyloxycarbonyl (FMOC) group, a benzyloxycarbonyl (Cbz or Z) group, a p-methoxybenzyl carbonyl (Moz or MeOZ) group, an allyloxycarbonyl (Alloc) group, a 2,2,2-Trichloroethoxycarbonyl (Troc) group, or a 2-(Trimethylsilyl)ethoxycarbonyl (Teoc) group.

62. The compound of claim 60, wherein at least one of R_{3b}, R_{4b}, R_{6b}, R_{7b}, and R_{8b} is not hydrogen.

63. The compound of claim 60, wherein at least one of R_{3b} or R_{4b} is not methyl.

64. The compound of claim 60, wherein:
R_{1b} is methyl;
R_{2b} is selected from hydrogen, methyl, and a protecting group;
R_{3b} is selected from hydroxyl, —OR_{9b}, —OC(O)R_{5b}, —OC(O)OR_{5b}, or —OSO₂R_{5b}; and
R_{4b}, R_{6b}, R_{7b}, and R_{8b} are all hydrogen;
with the proviso that R_{3b} is not hydroxyl when R_{2b} is methyl and R_{12b} is a tert-butyloxycarbonyl (BOC) group.

65. The compound of claim 55, wherein the tryptamine compound is a compound of formula (I).

66. N—BOC-Norpsilocin or a pharmaceutically-acceptable addition salt thereof.

67. N—BOC-4-Acetoxy-Norpsilocin or a pharmaceutically-acceptable addition salt thereof.

68. A composition comprising a compound of claim 55 or a pharmaceutically-acceptable acid addition salt thereof, and an excipient.

69. A composition comprising as a first active component: a compound of claim 55; and as a second active component selected from at least one of (a) a serotonergic drug, (b) a purified psilocybin derivative, (c) a purified cannabinoid, (d) a monoamine oxidase inhibitor, (e) a purified terpene, (f) an adrenergic drug, (g) a dopaminergic drug, (h) a purified erinacine, or (i) a purified hericenone; and a pharmaceutically acceptable excipient.

70. A method of preventing or treating a psychological disorder comprising:
identifying a subject in need of treatment or prevention; and
administering to a subject in need thereof a therapeutically effective amount of a compound of claim 55.

71. A method of generating a monoalkyltryptamine compound in situ in a patient, the method comprising administering to a patient a compound of claim 55.

72. A method of preventing or treating inflammation and/or pain comprising:
identifying a subject in need of treatment or prevention; and
administering to a subject in need thereof a therapeutically effective amount of a compound of claim 55.
